(19) United States
(12) Patent Application Publication
(10) Pub. No.: US 2015/0204829 A1
(43) Pub. Date: Jul. 23, 2015

(54) CHALCOGEN GAS MONITORING DEVICE

(71) Applicant: Electronics and Telecommunications Research Institute, Daejon (KR)

(72) Inventors: Daehyung CHO, Daejeon (KR); Yong-Duck CHUNG, Daejeon (KR)

(73) Assignee: Electronics and Telecommunications Research Institute, Daejon (KR)

(21) Appl. No.: 14/447,789

(22) Filed: Jul. 31, 2014

(30) Foreign Application Priority Data
Jan. 20, 2014 (KR) 10-2014-0006791

(51) Int. Cl. G01N 33/00; G01N 27/12; G01N 21/78

(52) U.S. Cl. CPC G01N 33/0036; G01N 21/783; G01N 27/125; G01N 27/12; G01N 33/0062; G01N 2033/0068

(57) ABSTRACT

Provided is a chalcogen gas monitoring device. The chalcogen gas monitoring device includes a reaction unit which varies in resistance due to reaction occurring by contact between chalcogen gas and a metal foil, a measurement unit measuring a resistance value according to the variation in resistance, a calculation unit measuring at least one of whether the chalcogen gas exists and a concentration of the chalcogen gas according to the resistance value, and a display unit outputting the measured results; wherein the metal foil is replaced according to results obtained by the reaction between the metal foil and the chalcogen gas.

### CROSS-REFERENCE TO RELATED APPLICATIONS

This U.S. non-provisional patent application claims priority under 35 U.S.C. §119 of Korean Patent Application No. 10-2014-0006791, filed on Jan. 20, 2014, the entire contents of which are hereby incorporated by reference.

### BACKGROUND OF THE INVENTION

The present invention disclosed herein relates to a monitoring device, and more particularly, to a chalcogen gas monitoring device for detecting whether a chalcogen gas exists.

The chalcogen materials are elements belong to the group 16 in the periodic table. For example, the chalcogen materials include oxygen (O), sulfur (S), selenium (Se), tellurium (Te), polonium (Po), and so on.

Chalcogen materials are combined with other elements to form compounds which are being widely used in the industrial fields. Here, for example, the chalcogen compounds include zinc oxide (ZnO), zinc sulfide (ZnS), zinc selenide (ZnSe), cadmium telluride (CdTe), CIGS (Cu(InGa)Se₂), indium trisulfide (In₂S₃), and so on. As one of the methods of forming the chalcogen compounds, there is a method in which a chalcogen element is evaporated in a gaseous form to react with a precursor. Here, it may be determined whether the chalcogen gas exists, or the chalcogen gas may be measured in concentration while the process for forming the chalcogen compound is performed. Thus, the chalcogen compound may have high quality.

For example, hydrocarbon fuel gas in a fuel cell contains sulfur (S) among the chalcogen materials, and the sulfur (S) leads to deterioration in performance of an anode and modified catalyst of the fuel cell. Therefore, it is necessary to detect and remove the sulfur (S) before the fuel gas is supplied. Also, since the chalcogen materials are highly toxic, there is a need to detect and block the leakage of the chalcogen materials in advance in the industrial site.

A quartz crystal monitor may be used to monitor the gaseous chalcogen material. However, the chalcogen material, especially in case of sulfur (S), does not easily stick to the surface of a quartz crystal.

Therefore, gas chromatography (GC) technologies are being used to monitor the concentration of the chalcogen materials. These gas chromatography technologies include a gas chromatography technology (GC-SCD or GC-FPD) related to sulfur chemiluminescence detection (SCD) or flame photometric detection (FPD). Although such a gas chromatography technology ensures superior monitoring performance of the chalcogen material, a large amount of costs is expended.

### SUMMARY OF THE INVENTION

The present invention provides a chalcogen gas monitoring device that is reduced in cost for monitoring chalcogen gas in a state where good monitoring performance of the chalcogen gas is ensured.

The present invention also provides a chalcogen gas monitoring device that is capable of determining whether chalcogen gas exists or monitoring a concentration of the chalcogen gas.

Embodiments of the present invention provide chalcogen gas monitoring devices including a reaction unit which varies in resistance due to reaction occurring by contact between chalcogen gas and a metal foil; a measurement unit measuring a resistance value according to the variation in resistance; a calculation unit measuring at least one of whether the chalcogen gas exists and a concentration of the chalcogen gas according to the resistance value; and a display unit outputting the measured results; wherein the metal foil is replaced according to results obtained by the reaction between the metal foil and the chalcogen gas.

In some embodiments, the chalcogen gas monitoring devices may further include an input unit receiving parameters for determining whether the chalcogen gas exists and measuring the concentration of the chalcogen gas according to a kind of metal foil to provide the parameters to the calculation unit.

In other embodiments, the parameters may include a kind of metal foil and a kind of chalcogen gas, and a reduced concentration according to the resistance value.

In still other embodiments, the measurement unit may output the measured resistance value to the display unit.

In even other embodiments, the reaction unit may include two metal electrodes that are in contact with the metal foil to output the resistance value due to the reaction with the chalcogen gas to the measurement unit.

In yet other embodiments, the metal electrodes may be formed of one of aluminum (Al), copper (Cu), iron (Fe), chrome (Cr), gold (Au), silver (Ag), and molybdenum (Mo).

In further embodiments, the reaction unit may further include: a support layer supporting the metal electrodes and the metal foil; and a cover protecting the metal foil, the metal electrodes, and the support layer, the cover having an opened portion to allow the metal foil to contact the chalcogen gas.

In still further embodiments, the reaction unit may further include a metal detection sensor detecting at least one of electrical characteristics and chemical characteristics of the metal foil.

In even further embodiments, the chalcogen gas monitoring devices may further include a metal foil determination unit determining a kind of metal foil by using information detected through the metal detection sensor.

In yet further embodiments, the chalcogen gas monitoring devices may further include an input unit receiving parameters for determining whether the chalcogen gas exists and measuring the concentration of the chalcogen gas according to a kind of metal foil to provide the parameters to the calculation unit.

In much further embodiments, the parameters may include a kind of chalcogen gas and a reduced concentration according to the resistance value.

In still much further embodiments, the metal foil may be formed of one of copper (Cu), zinc (Zn), molybdenum (Mo), aluminum (Al), titanium (Ti), tin (Sn), nickel (Ni), and chrome (Cr).

In even much further embodiments, the calculation unit may output information for instructing the replacement of the metal foil to the display unit when the resistance value does not exceed a preset value.

In yet much further embodiments, the reaction unit may further include a color sensor for detecting a change in color of the metal foil due to the reaction with the chalcogen gas.

In much still further embodiments, the measurement unit may measure the color change, the calculation unit measuring at least one of whether the chalcogen gas exists and the concentration of the chalcogen gas according to the color change.

In other embodiments of the present invention, chalcogen gas monitoring devices include: a reaction unit which varies in resistance due to reaction with chalcogen gas; a measurement unit measuring a resistance value according to the variation in resistance; a calculation unit measuring at least one of whether the chalcogen gas exists and a concentration of the chalcogen gas according to the resistance value; and a display unit outputting the measured results, wherein the reaction unit includes: a metal foil replaced according to the results obtained by the reaction with the chalcogen gas; and two metal electrodes that is in contact with the metal foil to output the resistance value due to the reaction with the chalcogen gas to the measurement unit.

In some embodiments, the reaction unit may include: a support layer supporting the metal electrodes and the metal foil; and a cover protecting the metal foil, the metal electrodes, and the support layer, the cover having an opened portion to allow the metal foil to contact the chalcogen gas.

In still other embodiments of the present invention, chalcogen gas monitoring devices include: a reaction unit which varies in resistance due to reaction with chalcogen gas; a measurement unit measuring a resistance value according to the variation in resistance; a metal foil determination unit determining a metal material of the metal foil by at least one of electrical characteristics and chemical characteristics of the metal foil; a calculation unit measuring at least one of whether the chalcogen gas exists and a concentration of the chalcogen gas according to the resistance value and the determined metal material; and a display unit outputting the measured results, wherein the reaction unit include: a metal foil replaced according to the results obtained by the reaction with the chalcogen gas; two metal electrodes that is in contact with the metal foil to output the resistance value due to the reaction with the chalcogen gas to the measurement unit; and a metal detection sensor detecting at least one of the electrical characteristics and the chemical characteristics of the metal foil.

In some embodiments, the reaction unit may include: a support layer supporting the metal electrodes, the metal foil, and the metal detection sensor; and a cover protecting the metal foil, the metal electrodes, and the support layer, the cover having an opened portion to allow the metal foil to contact the chalcogen gas.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a view of a chalcogen gas monitoring device according to the present invention;

FIG. 2 is a front view of a reaction unit of the chalcogen gas monitoring device illustrated in FIG. 1;

FIG. 3 is a cross section diagram of the reaction unit of the chalcogen gas monitoring device illustrated in FIG. 2;

FIG. 4 is a graph illustrating a variation in resistance of a metal foil depending on a kind of chalcogen gas according to an embodiment of the present invention;

FIG. 5 is a view illustrating another example of the chalcogen gas monitoring device according to the present invention;

FIG. 6 is a front view of a reaction unit of the chalcogen gas monitoring device illustrated in FIG. 5; and

FIG. 7 is a cross section diagram of the reaction unit of the chalcogen gas monitoring device illustrated in FIG. 6.

### DETAILED DESCRIPTION OF PREFERRED EMBODIMENTS

Hereinafter, it will be described about an exemplary embodiment of the present invention in conjunction with the accompanying drawings. Therefore, in some embodiments, well-known processes, device structures, and technologies will not be described in detail to avoid ambiguousness of the present invention. In following descriptions, it is noted that only portions that is necessary for an understanding of operations according to the present invention will be described, and descriptions with respect to the rest portions will be omitted to avoid ambiguous interpretation of the present invention.

The present invention provides a chalcogen gas monitoring device that is capable of determining whether chalcogen gas exists or monitoring a concentration of the chalcogen gas. The chalcogen gas monitoring device may use a thin film metal foil formed of a metal material for reacting with the chalcogen gas. Here, the chalcogen gas is composed of chalcogen materials that are elements belong to the group 16 in the periodic table. For example, the chalcogen materials include oxygen (O), sulfur (S), selenium (Se), tellurium (Te), polonium (Po), and so on.

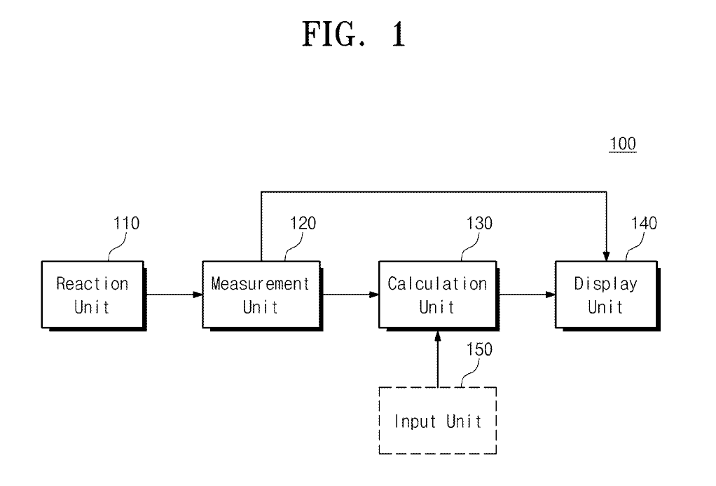

FIG. 1 is a view of a chalcogen gas monitoring device according to the present invention.

Referring to FIG. 1, the chalcogen gas monitoring device 100 includes a reaction unit 110, a measurement unit 120, a calculation unit 130, and a display unit 140. Also, the chalcogen gas monitoring device 100 may further include an input unit 150.

The reaction unit 110 reacts with the chalcogen gas to vary in resistance. For this, the reaction unit 110 includes a metal foil for reacting with the chalcogen gas. Here, the metal foil is a metal material for reacting with the chalcogen gas. For example, the metal foil may be formed of a metal material such as copper (Cu), zinc (Zn), molybdenum (Mo), aluminum (Al), titanium (Ti), tin (Sn), nickel (Ni), and chrome (Cr) that react with the chalcogen gas. When the metal foil is completely used through the reaction with the chalcogen gas, the metal foil may be replaced. A metal—chalcogen compound generated by the reaction between the metal material and the chalcogen gas. The metal-chalcogen compound has resistance greater than that of the metal material. Thus, the reaction unit 110 may use a variation in characteristic of the metal-chalcogen compound that is generated due to the reaction between the metal material and the chalcogen gas.

The measurement unit 120 measures a resistance value according to a variation in resistance of the metal foil that reacts with the chalcogen gas. The measurement unit 120 outputs the measured resistance value to the calculation unit 130. Also, the measurement unit 120 may output the measured resistance value to the display unit 140.

The calculation unit 130 may receive the resistance value through the measurement unit 120. Here, the calculation unit 130 may store parameters for determining whether the chalcogen gas exists and for measuring a concentration of the chalcogen gas, or may receive parameters from the outside. The parameters may include kinds of metal foil and chalcogen gas, a reduced concentration depending on the resistance value (or a constant for calculating the resistance value), and so on.

The calculation unit 130 may determine whether the chalcogen gas exists or calculate a concentration of the chalcogen gas by using the parameters according to the received resistance value. The calculation unit 130 outputs the calculated result to the display unit 140. When the calculation unit 130 detects no change in resistance value due to the complete reaction between the chalcogen gas and the metal foil through the variation in resistance received through the measurement unit 120, the calculation unit 130 may output information for informing replacement of the metal foil to the display unit 140.

The display unit 140 may include a display device for displaying the resistance value to inform the resistance value to a user. The display unit 140 displays the resistance value received through the measurement unit 120. Also, the display unit 140 displays the existence/nonexistence of the chalcogen gas or the concentration of the chalcogen gas which are outputted through the calculation unit 130.

The input unit 150 receives the parameters for monitoring the chalcogen gas from the user to output the received parameter information to the calculation unit 130.

The display unit 140 may output the replacement information of the metal foil or display information for inputting the parameters.

The chalcogen gas monitoring device 100 of the present invention may be used at atmospheric pressure (about 1 atm) or in a vacuum state in which a pressure is less than the atmospheric pressure.

Thus, the chalcogen gas monitoring device 100 of the present invention may determine whether the chalcogen gas exists and to measure the concentration of the chalcogen gas by using the metal foil reacting with the chalcogen gas. The chalcogen gas monitoring device 100 may replace the metal foil that completely reacted with the chalcogen gas. Therefore, the chalcogen gas monitoring device 100 may ensure uniform performance and may reduce costs consumed to detect the chalcogen gas when compared to the existing gas chromatography technology.

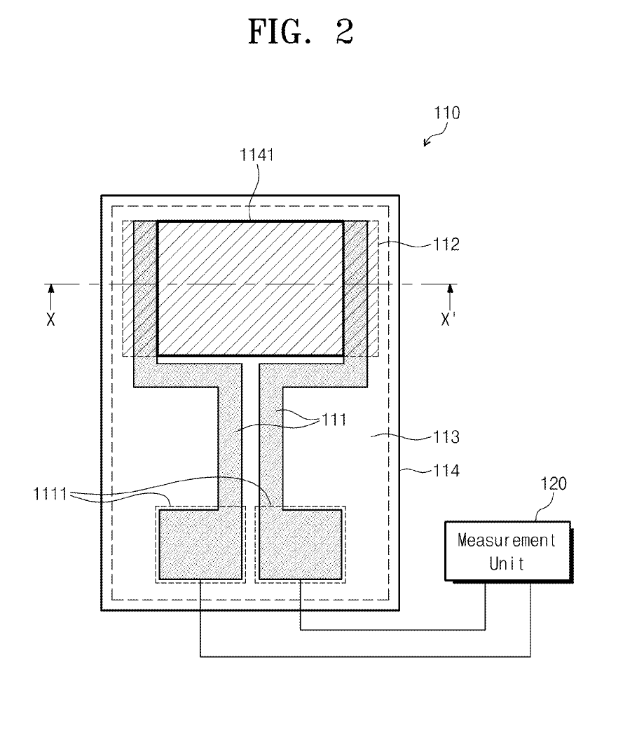

FIG. 2 is a front view of a reaction unit of the chalcogen gas monitoring device illustrated in FIG. 1.

Referring to FIG. 2, the reaction unit 110 includes metal electrodes 111, the metal foil 112, a support layer 113, and a cover 114.

The metal electrodes 111 may be formed of a high-conductive metal material, for example, aluminum (Al), copper (Cu), iron (Fe), chrome (Cr), gold (Au), silver (Ag), and molybdenum (Mo). A portion of each of the metal electrodes 111 may be in contact with the metal foil 112. A portion of each of the metal electrodes 111 may be provided in the form of a contact pad 1111 to be connected the measurement unit 120.

The metal foil 112 reacts with the chalcogen gas. For example, the metal foil 112 may be formed of a metal material such as copper (Cu), zinc (Zn), molybdenum (Mo), aluminum (Al), titanium (Ti), tin (Sn), nickel (Ni), and chrome (Cr) that react with the chalcogen gas. The metal foil 112 may have a thin film shape. For example, the metal foil 112 may have a thin film shape with a thickness of about 1 μm to about 3 mm. Also, when the metal foil 112 completely reacts with the chalcogen gas, (for example, when the resistance is no longer increasing) the metal foil 112 is replaced.

The support layer 113 has a function for supporting the metal electrodes 111 and the metal foil 112. The support layer 113 may be formed of a non-conductive material, for example, a material such as a kind of plastic, a kind of glass.

The cover 114 may protect the metal electrodes 111 and the support layer 113 against the chalcogen gas and fix the metal foil 112. Also, the cover 114 may have an upper portion with a portion opened to allow the metal foil 112 to react with the chalcogen gas. For example, an opened area 114 in which the metal foil 112 reacts with the chalcogen gas is defined in the cover 114.

The cover 114 has an openable structure so as to replace the metal foil 112. The cover 114 may be formed of a material such as glasses, plastics, and metals.

Thus, when the metal foil 112 is completely used, the user may open the cover 114 to remove the used metal foil 112. Thereafter, a new metal foil is mounted, and then the cover 114 is closed to monitor the chalcogen gas again.

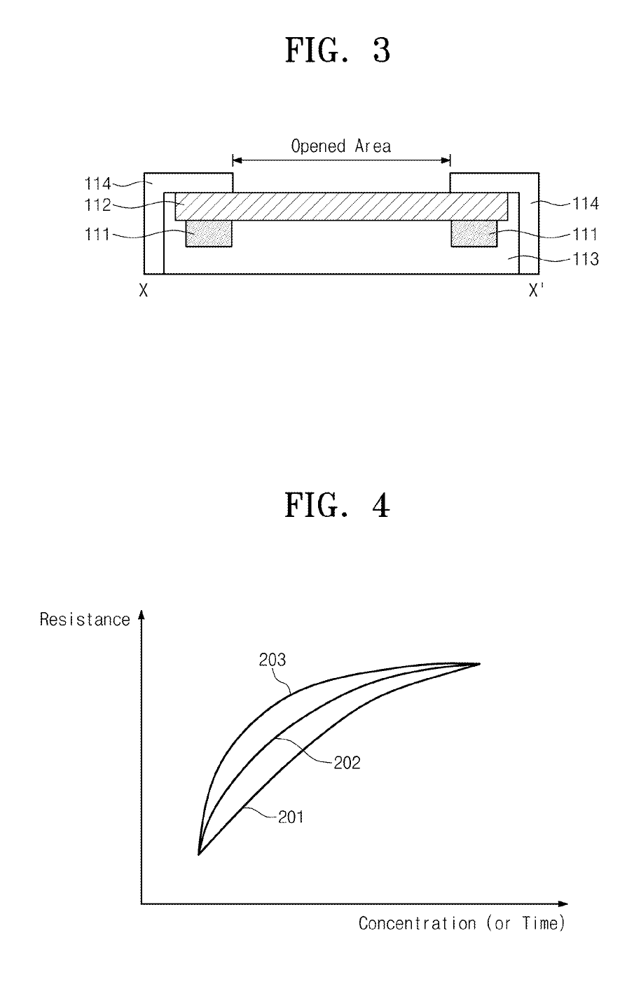

FIG. 3 is a cross section diagram of the reaction unit of the chalcogen gas monitoring device illustrated in FIG. 2.

Referring to FIG. 3, a cross section of the reaction unit 110 that is taken along line X-X′ of FIG. 2 is illustrated. The reaction unit 110 includes the metal electrodes 111, the metal foil 112, the support layer 113, and the cover 114.

The metal electrodes 111 are disposed between the metal foil 112 and the support layer 113 to contact the metal foil 112.

The metal foil 112 is disposed on the metal electrodes 111.

The support layer 113 is disposed on lower ends of the metal electrodes 111 and the metal foil 112.

The cover 114 surrounds the metal electrode 111, the metal foil 112, and the support layer 113 to protect the metal electrode 111, the metal foil 112, and the support layer 113. The cover 114 includes the opened area for the reaction of the metal foil 112 with the chalcogen gas.

FIG. 4 is a graph illustrating a variation in resistance of a metal foil depending on a kind of chalcogen gas according to an embodiment of the present invention.

Referring to FIG. 4, an abscissa of the graph represents a concentration or time, and an ordinate represents resistance. Here, reference numeral 201 represents a variation in resistance of the metal material that reacts with selenium (Se), and reference numeral 202 represents a variation in resistance of the metal material that reacts with sulfur (S). Also, reference numeral 203 represents a variation in resistance of the metal material that reacts with tellurium (Te).

As described above, in the chalcogen gas monitoring device 100 of the present invention, when the metal foil 112 that is a metal material reacts with the chalcogen material, the metal-chalcogen compound is formed.

For example, when copper (Cu) that is a metal material reacts with sulfur (S) that is a chalcogen material, copper sulfide (CuₓS_{y}) is formed. Also, when zinc (Zn) that is a metal material reacts with sulfur (S) that is a chalcogen material, zinc sulfide (ZnS) is formed. Zinc sulfide (ZnS) and copper sulfide (CuS) are semiconductor materials and have resistance values greater than those of copper (Cu) and sulfur (S).

Thus, when the chalcogen increases in concentration, or as time passes, the metal material exposed to the chalcogen material gradually changes into the compound, and the metal material also changes in resistance.

The chalcogen gas monitoring device 100 may determine whether the chalcogen gas exists and may measure a concentration of the chalcogen gas through the variation in resistance according to a kind of chalcogen, a kind of metal foil, a concentration of the chalcogen, and an exposure time.

Also, the reaction unit 110 of the chalcogen gas monitoring device 100 of the present invention may include a color sensor that is capable of detecting a change in color of the metal-chalcogen compounds. Here, since the metal reacting with the chalcogen compounds changes in color, the measurement unit 120 connected to the color sensor may measure a change in color. The calculation unit 130 may determine whether the chalcogen gas exists and may measure a concentration of the chalcogen gas on the basis of the color change measured by the measurement unit 120.

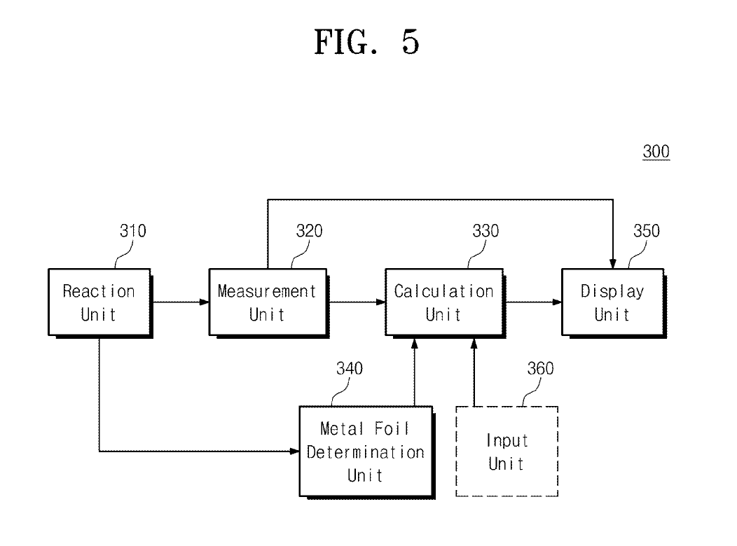

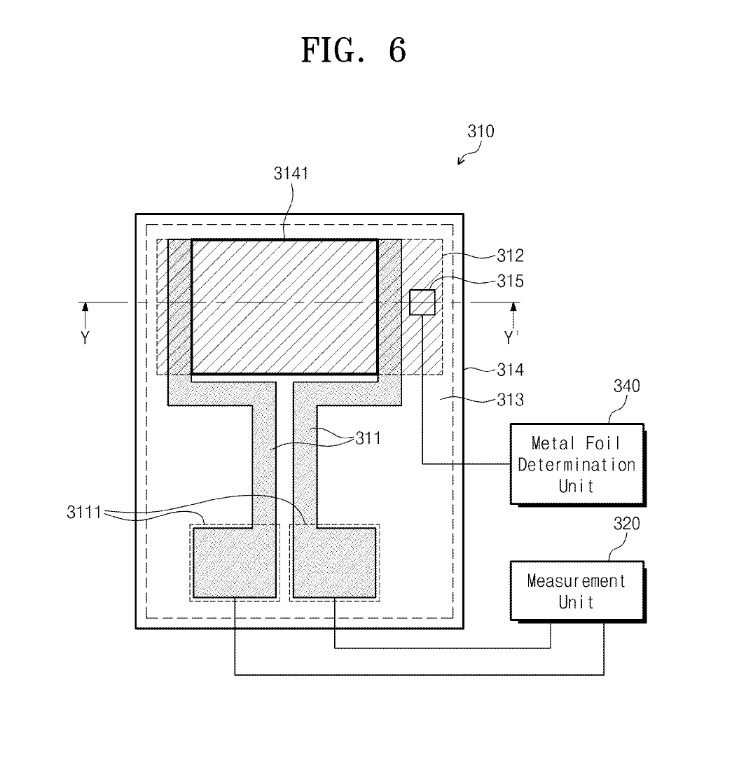

FIG. 5 is a view illustrating another example of the chalcogen gas monitoring device according to the present invention.

Referring to FIG. 5, a chalcogen gas monitoring device 300 includes a reaction unit 310, a measurement unit 320, a calculation unit 330, a metal foil determination unit 340, and a display unit 350. Also, the chalcogen gas monitoring device 300 may further include an input unit 360.

The chalcogen gas monitoring device 300 is generally similar to the chalcogen gas monitoring device 100 of FIG. 1 except for a change in structure due to the metal foil determination unit 340. Therefore, configurations similar to those of the chalcogen gas monitoring device 100 will be described in detail with reference to FIG. 1.

As a replaceable metal foil is used, the chalcogen gas monitoring device 300 may further include the metal foil determination unit 340.

Here, the reaction unit 310 may further include a metal detection sensor for detecting a kind of metal foil. The metal detection sensor may detect the kind of metal foil by using electrical characteristics or chemical characteristics of the metal foil.

The reaction unit 310 may output electrical characteristic information or chemical characteristic information of the metal foil, which are measured through the metal detection sensor, to the metal foil determination unit 340. Various methods may be applied to detect the metal material constituting the metal foil. For example, the metal detect sensor may detect the metal material through the electrical characteristic information such as electrical conductivity, a thermal electromotive force, and so on.

Alternatively, information with respect to the metal material constituting the metal foil may be recorded in the metal foil, and thus the metal detection sensor of the reaction unit 310 may read and detect the information with respect to the metal material from the metal foil.

The reaction unit 310 outputs the characteristic information or read information with respect to the metal foil to the metal foil determination unit 340.

The metal foil determination unit 340 may determine a kind of metal foil that is attached to the chalcogen gas monitoring device 300 by using the characteristic information and the read information with respect to the metal foil. The metal foil determination unit 340 outputs the information with respect to the determined metal foil to the calculation unit 330.

Therefore, even if the input unit 360 does not input the information with respect to the metal foil into the calculation unit 330, the calculation unit 330 may acquire the information with respect to the metal foil from the metal foil determination unit 340.

FIG. 6 is a front view of a reaction unit of the chalcogen gas monitoring device illustrated in FIG. 5. and

Referring to FIG. 6, the reaction unit 310 includes metal electrodes 311, the metal foil 312, a support layer 313, a cover 314, and the metal detection sensor 315.

Here, the configurations of the reaction unit 310 except for the metal detection sensor 315 will be described in detail with reference to FIG. 2.

For example, the metal detection sensor 315 may be in contact with the metal foil 312 on a cross section of each of the metal electrodes. The metal detection sensor 315 may detect electrical characteristics, chemical characteristics of the metal foil 312, and information recorded in the metal foil.

The metal detection sensor 315 is connected to the metal foil determination unit 340 to output the detected information to the metal foil determination unit 340.

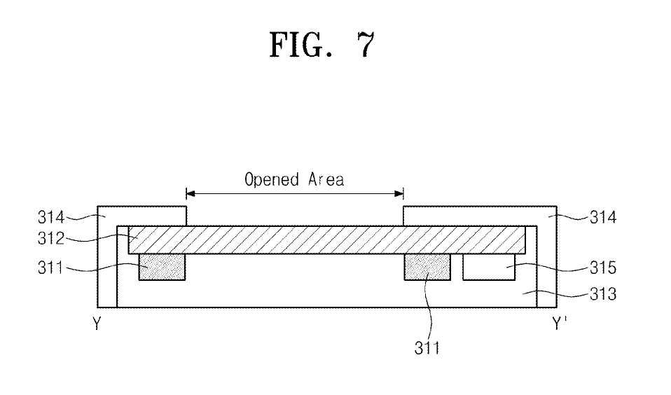

FIG. 7 is a cross section diagram of the reaction unit of the chalcogen gas monitoring device illustrated in FIG. 6.

Referring to FIG. 7, a cross section of the reaction unit 310 that is taken along line Y-Y′ of FIG. 6 is illustrated. The reaction unit 310 includes metal electrodes 311, the metal foil 312, the support layer 313, the cover 314, and the metal detection sensor 315.

Here, the configurations of the reaction unit 310 except for the metal detection sensor 315 will be described in detail with reference to FIG. 3.

The metal detection sensor 315 is disposed between the metal foil 312 and the support layer 313. The metal detection sensor 315 is disposed on a lower end of the metal foil 312 and an upper end of the support layer 313.

Also, the metal detection sensor 315 is disposed in the cover.

The position of the metal detection sensor described in FIGS. 6 and 7 is exemplarily described, for example the metal detection sensor may be positioned at various positions for detecting the metal material constituting the metal foil 312.

The chalcogen gas monitoring device 300 provided in FIGS. 5 to 7 may directly detect a kind of replaced metal and may be used to monitor the chalcogen gas. Thus, it is unnecessary to input the information due to the replacement of the metal foil by a user.

As described above, the chalcogen gas monitoring device proposed in the present invention may use the detachable metal foil as a reaction instructor of the chalcogen gas to monitor whether the chalcogen gas exists. In addition, the chalcogen gas monitoring device may also monitor the concentration of the chalcogen gas. Therefore, the chalcogen gas monitoring device of the present invention may simply monitor the chalcogen gas at low costs.

The chalcogen gas monitoring device of the present invention may use the replaceable metal foil to monitor the chalcogen gas, thereby reducing costs required for monitoring the chalcogen gas in a state where good monitoring performance of the chalcogen gas is ensured. Also, the chalcogen gas monitoring device of the present invention may allow the metal foil to react with the chalcogen gas to measure a variation in resistance, thereby determining whether the chalcogen gas exists or monitoring the concentration of the chalcogen gas.

Although specific embodiments are described in the detailed description of the inventive concept, the detailed description may be amended or modified without being out of the scope of the inventive concept. Therefore, the scope of the invention is defined not by the detailed description of the invention but by the appended claims, and all differences within the scope will be construed as being included in the present invention.

What is claimed is:

1. A chalcogen gas monitoring device comprising:
a reaction unit which varies in resistance due to reaction occurring by contact between chalcogen gas and a metal foil;
a measurement unit measuring a resistance value according to the variation in resistance;
a calculation unit measuring at least one of whether the chalcogen gas exists and a concentration of the chalcogen gas according to the resistance value; and
a display unit outputting the measured results;
wherein the metal foil is replaced according to results obtained by the reaction between the metal foil and the chalcogen gas.

2. The chalcogen gas monitoring device of claim 1, further comprising an input unit receiving parameters for determining whether the chalcogen gas exists and measuring the concentration of the chalcogen gas according to a kind of metal foil to provide the parameters to the calculation unit.

3. The chalcogen gas monitoring device of claim 2, wherein the parameters comprise a kind of metal foil and a kind of chalcogen gas, and a reduced concentration according to the resistance value.

4. The chalcogen gas monitoring device of claim 1, wherein the measurement unit outputs the measured resistance value to the display unit.

5. The chalcogen gas monitoring device of claim 1, wherein the reaction unit comprises two metal electrodes that is in contact with the metal foil to output the resistance value due to the reaction with the chalcogen gas to the measurement unit.

6. The chalcogen gas monitoring device of claim 5, wherein the metal electrodes are formed of one of aluminum (Al), copper (Cu), iron (Fe), chrome (Cr), gold (Au), silver (Ag), and molybdenum (Mo).

7. The chalcogen gas monitoring device of claim 5, wherein the reaction unit further comprises:
a support layer supporting the metal electrodes and the metal foil; and
a cover protecting the metal foil, the metal electrodes, and the support layer, the cover having an opened portion to allow the metal foil to contact the chalcogen gas.

8. The chalcogen gas monitoring device of claim 5, wherein the reaction unit further comprises a metal detection sensor detecting at least one of electrical characteristics and chemical characteristics of the metal foil.

9. The chalcogen gas monitoring device of claim 8, further comprising a metal foil determination unit determining a kind of metal foil by using information detected through the metal detection sensor.

10. The chalcogen gas monitoring device of claim 9, further comprising an input unit receiving parameters for determining whether the chalcogen gas exists and measuring the concentration of the chalcogen gas according to a kind of metal foil to provide the parameters to the calculation unit.

11. The chalcogen gas monitoring device of claim 10, wherein the parameters comprise a kind of chalcogen gas and a reduced concentration according to the resistance value

12. The chalcogen gas monitoring device of claim 1, wherein the metal foil is formed of one of copper (Cu), zinc (Zn), molybdenum (Mo), aluminum (Al), titanium (Ti), tin (Sn), nickel (Ni), and chrome (Cr).

13. The chalcogen gas monitoring device of claim 1, wherein the calculation unit outputs information for instructing the replacement of the metal foil to the display unit when the resistance value does not exceed a preset value.

14. The chalcogen gas monitoring device of claim 1, wherein the reaction unit further comprises a color sensor for detecting a change in color of the metal foil due to the reaction with the chalcogen gas.

15. The chalcogen gas monitoring device of claim 14, wherein the measurement unit measures the color change,
the calculation unit measuring at least one of whether the chalcogen gas exists and the concentration of the chalcogen gas according to the color change.

16. A chalcogen gas monitoring device comprising:
a reaction unit which varies in resistance due to reaction with chalcogen gas;
a measurement unit measuring a resistance value according to the variation in resistance;
a calculation unit measuring at least one of whether the chalcogen gas exists and a concentration of the chalcogen gas according to the resistance value; and
a display unit outputting the measured results,
wherein the reaction unit comprises:
a metal foil replaced according to the results obtained by the reaction with the chalcogen gas; and
two metal electrodes that are in contact with the metal foil to output the resistance value due to the reaction with the chalcogen gas to the measurement unit.

17. The chalcogen gas monitoring device of claim 16, wherein the reaction unit comprises:
a support layer supporting the metal electrodes and the metal foil; and
a cover protecting the metal foil, the metal electrodes, and the support layer, the cover having an opened portion to allow the metal foil to contact the chalcogen gas.

18. A chalcogen gas monitoring device comprising:
a reaction unit which varies in resistance due to reaction with chalcogen gas;
a measurement unit measuring a resistance value according to the variation in resistance;
a metal foil determination unit determining a metal material of the metal foil by at least one of electrical characteristics and chemical characteristics of the metal foil;
a calculation unit measuring at least one of whether the chalcogen gas exists and a concentration of the chalcogen gas according to the resistance value and the determined metal material; and
a display unit outputting the measured results,
wherein the reaction unit comprises:
a metal foil replaced according to the results obtained by the reaction with the chalcogen gas;
two metal electrodes that is in contact with the metal foil to output the resistance value due to the reaction with the chalcogen gas to the measurement unit; and
a metal detection sensor detecting at least one of the electrical characteristics and the chemical characteristics of the metal foil.

19. The chalcogen gas monitoring device of claim 18, wherein the reaction unit comprises:
a support layer supporting the metal electrodes, the metal foil, and the metal detection sensor; and
a cover protecting the metal foil, the metal electrodes, and the support layer, the cover having an opened portion to allow the metal foil to contact the chalcogen gas.
